(19) United States
(12) Patent Application Publication
(10) Pub. No.: US 2015/0366767 A1
(43) Pub. Date: Dec. 24, 2015

(54) NON-STAINING TOOTHPASTE

(71) Applicant: COLGATE-PALMOLIVE COMPANY, New York, NY (US)

(72) Inventors: Gregory Szewczyk, Flemington, NJ (US); Neeta Atul Patel, Monmouth Junction, NJ (US); Suzanne Jogun, Wayne, NJ (US)

(73) Assignee: Colgate-Palmolive Company, Piscataway, NJ (US)

(21) Appl. No.: 14/654,671

(22) PCT Filed: Dec. 21, 2012
(86) PCT No.: PCT/US2012/071137

§ 371 Date: Jun. 22, 2015

(51) Int. Cl. A61K 8/29; A61K 8/49; A61K 8/46; A61K 8/25; A61K 8/34; A61K 8/21; A61K 8/73; A61Q 11/00; A61K 8/81

(52) U.S. Cl. CPC A61K 8/29; A61Q 11/00; A61K 8/494; A61K 8/463; A61K 8/8164; A61K 8/347; A61K 8/21; A61K 8/345; A61K 8/73; A61K 8/25; A61K 2800/58; A61K 2800/43

(57) ABSTRACT

The present invention relates to a dentifrice which does not excessively stain toothbrush bristles, for example, a pigmented toothpaste formulations having a pigment system which comprises (i) a dissolvable or disintegratable film comprising one or more releasable pigments which are released during brushing, and (ii) titanium dioxide, wherein the level of titanium dioxide is 0.01-0.375% by weight of the toothpaste; together with methods of making and using the same.

### BACKGROUND OF THE INVENTION

It is recommended that children should brush their teeth for at least 45-60 seconds, and adults for at least 90 to 120 seconds. Most people, especially children, do not brush their teeth for a sufficient period of time to obtain maximum benefit, and moreover have difficulty accurately estimating the time necessary to brush the teeth. Toothpaste formulations have been developed which comprise dissolvable films or microcapsules comprising pigment, which are designed to release the pigment after 30 to 120 seconds of brushing, providing a color change signal of adequate brushing. One problem with these formulations, however, is the tendency of the pigment to stain the bristles of the toothbrush. There is a need for improved formulations with reduced potential to stain the bristles, but still providing a clear signal upon adequate brushing.

### BRIEF SUMMARY OF THE INVENTION

Following systematic testing, varying different formulation components, we surprisingly found that an interaction exists between the polymer thickener (in this case Gantrez) and titanium dioxide (a common opaque white pigment) that drives the deposit of the colored (blue) pigment on the bristles. Unexpectedly, the lower the TiO₂ level, the less the blue pigment appears to deposit on the bristles. Without being bound by theory, we hypothesize that this is driven by a charge (polarization) phenomena.

The invention thus provides a pigment system for a dentifrice, wherein the dentifrice comprises a pigment and titanium dioxide, wherein the level of titanium dioxide is 0.01-0.375% by weight of the dentifrice.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION OF THE INVENTION

The following description of the preferred embodiment(s) is merely exemplary in nature and is in no way intended to limit the invention, its application, or uses.

The invention thus provides, in a first embodiment, a dentifrice (Dentifrice 1) having a pigment system which comprises one or more pigments and titanium dioxide, wherein the level of titanium dioxide is 0.01-0.375% by weight of the dentifrice; for example:
1.1. Dentifrice 1 wherein the one or more pigments is selected from one or more insoluble pigments, e.g., a red pigment, for example D&C Red 30, a green pigment, for example Pigment Green 7, a yellow pigment, e.g. (Natpure LC 128 Yellow, from Sensient Co.), and a blue pigment, for example a phthalocyanine, for example Pigment Blue 15:

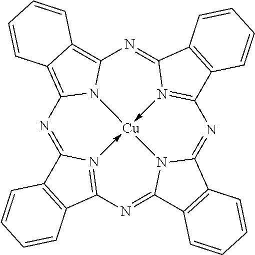

1.2. In one embodiment, the one or more pigments are present in an amount from about 0.02 to about 0.5% of the total weight of the composition. In one embodiment, the one or more pigments are present in an amount from about 0.05 to about 0.3% of the total weight of the composition.
1.3. Any of the foregoing dentrifices wherein the pigment is Pigment Blue 15.
1.4. Any of the foregoing dentifrices wherein the one or more pigments are releasable during brushing with an oral care implement. In another embodiment of this dentifrice, a dissolvable or disintegratable film comprises the one or more releasable pigments which are released during brushing.
1.5. Any of the foregoing dentifrices wherein the releasable pigment provides a color signal to the user of adequate brushing and the titanium dioxide reduces or inhibits the staining of the oral care implement by the releasable pigment
1.6. Any of the foregoing dentifrices wherein the oral care implement is a toothbrush.
1.7. Any of the foregoing dentifrices wherein the amount of titanium dioxide is 0.05-0.375%, e.g. 0.05 to 0.2, e.g., about 0.1%.
1.8. Any of the foregoing dentifrices comprising a copolymer of methyl vinyl ether and maleic anhydride having a molecular weight (M.W.) of about 30,000 to about 1,000,000, most preferably about 30,000 to about 800,000 (for example as sold under the tradename Gantrez® e.g., AN 139 (M.W. 500,000), AN 119 (M.W. 250,000) and preferably S-97 Pharmaceutical Grade (M.W. 700,000) available from ISP Technologies, Inc., Bound Brook, N.J. 08805), e.g., in an amount of 1-3%, e.g., about 2%.
1.9. Any of the foregoing dentifrices comprising 1.5-2.0% of sodium lauryl sulfate and 0.5-2.0% of a copolymer of methyl vinyl ether and maleic anhydride.
1.10. Any of the foregoing dentifrices wherein the dentifrice further comprises an effective amount of an antibacterial agent, e.g., triclosan, e.g., about 0.3%; an anionic surfactant, e.g., sodium lauryl sulfate (SLS), e.g. 1.5-2%, an effective amount of a fluoride source, e.g., a soluble fluoride salt, e.g. sodium fluoride, about 0.24%; humectants, e.g. comprising one or more of glycerin, sorbitol, and propylene glycol, e.g., 45-75%; thickeners, e.g., selected from xanthan gum, carrageenan, and colloidal silica, e.g., 1-10%; an anionic polymer, e.g., a copolymer of methyl vinyl ether and maleic anhydride (for example, Gantrez®), e.g., 0.5-3%, e.g. about 2%, and water, e.g., 15-25%, e.g., about 20%.
1.11. Any of the foregoing dentifrices wherein the releasable pigment is substantially released after a period of greater than 30 seconds and less than 180 seconds of brushing in the presence of water, e.g., about 45-60 seconds in a toothpaste for use by a child and about 90-120 seconds in a toothpaste for use by an adult; wherein the release of the releasable pigment provides a color signal to the user of adequate brushing.
1.12. Any of the foregoing dentifrices wherein the releasable pigment does not significantly stain the bristles of the toothbrush during use.

The invention further provides a method of cleaning the teeth comprising brushing with a dentifrice having a pigment system which comprises (i) a dissolvable or disintegratable film comprising one or more releasable pigments which are released during brushing, and (ii) titanium dioxide, wherein the level of titanium dioxide is 0.01-0.375%, e.g., about 0.1% by weight of the dentifrice, e.g., any of Dentifrice 1, et seq., for example
a. the method wherein the brushing is continued until the film disintegrates and the pigment provides a color signal to the user of adequate brushing, for example,
b. the foregoing method when the brushing time before the film disinegrates is between 30 and 180 seconds, e.g., about 45-60 seconds for a toothpaste for use by a child and about 90-120 seconds for a toothpaste for use by an adult.

Orally acceptable: The compositions of the invention are intended for topical use in the mouth, thus components for use in the present invention should be orally acceptable, that is, safe for topical use in the mouth, in the amounts and concentrations provided.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

Embodiments of the present invention are further described in the following examples. The examples are merely illustrative and do not in any way limit the scope of the invention as described and claimed.

### EXAMPLES

### Example 1

### Formulation Optimization

In initial experiments wherein toothpaste was prepared by admixing dissolvable film flakes comprising Pigment Blue 15 in a modified commercial dentifrice base, the toothbrush bristles are stained blue following repeated use and release of the pigment. The amount of blue on the bristles is measured using Lab color space (a color-opponent space with dimension L for lightness and a and b for the color-opponent dimensions, based on nonlinearly compressed CIE XYZ color space coordinates).

Initial experiments are performed with a variety of formulations to determine the key formulation drivers of bristle staining. A Design of Experiments approach is utilized to refine and systematically optimize the formulation, focusing on three ingredients.

Twenty-five formulations were prepared, with variables within this framework, and are systematically assessed for performance and bristle staining. The formulations containing Gantrez (a copolymer of methyl vinyl ether and maleic anhydride), sodium lauryl sulfate (SLS) and titanium dioxide within the above ranges follow below in Table 1:

TABLE 1

| Formulations | % w/w |
|---|---|
| GANTREZ ® S-97 (a copolymer of methyl | 0.50-2.00 |
| vinyl ether and maleic anhydride) | |
| TITANIUM DIOXIDE | 0.01-0.375 |
| SODIUM LAURYL SULFATE (SLS) | 1.50-2.00 |
| WATER | 18.10 |
| SODIUM FLUORIDE | 0.24 |
| SODIUM SACCHARIN | 0.30 |
| GLYCERIN | 29.73-32.11 |
| XANTHAN GUM | 0.50 |
| IOTA CARRAGEENAN | 0.50 |
| SORBITOL | 20.85 |
| PROPYLENE GLYCOL | 0.50 |
| NaOH, 50% SOLUTION | 1.20 |
| DENTAL TYPE SILICA-ZEODENT 105-HIGH | 10.00 |
| CLEANING SILICA | |
| DENTAL TYPE SILICA (ZEODENT 115) | 8.50 |
| ABRASIVE | |
| DENTAL TYPE SILICA-ZEODENT 165-SYNTH. | 3.00 |
| AMORPHOUS | |
| MICA | 0.70 |
| FILM COMPRISING PIGMENT BLUE 15 | 0.20 |
| TRICLOSAN | 0.30 |
| FLAVOR | 1.00 |
| Total | 100.00 |

It is surprisingly found that an interaction exists between Gantrez (a copolymer of methyl vinyl ether and maleic anhydride) and titanium dioxide that can cause deposit of the blue pigment on the bristles. Unexpectedly, the lower the TiO₂ level, the less the blue pigment appears to deposit on the bristles. It is hypothesized that deposition may be driven by a charge (polarization) phenomena.

An optimized formulation is prepared, based on this data in Table 2 below:

TABLE 2

| | % w/w |
|---|---|
| GANTREZ S-97 (a copolymer of methyl | 2.0 |
| vinyl ether and maleic anhydride) | |
| TITANIUM DIOXIDE | 0.1 |
| SODIUM LAURYL SULFATE (SLS) | 1.75 |
| WATER | 18.10 |
| SODIUM FLUORIDE | 0.24 |
| SODIUM SACCHARIN | 0.30 |
| GLYCERIN | 32.11 |
| XANTHAN GUM | 0.50 |
| IOTA CARRAGEENAN | 0.50 |
| SORBITOL | 20.85 |
| PROPYLENE GLYCOL | 0.50 |
| NaOH, 50% SOLUTION | 1.20 |
| DENTAL TYPE SILICA (ZEODENT 105) | 10.00 |
| HIGH CLEANING SILICA | |
| DENTAL TYPE SILICA (ZEODENT 115) | 8.50 |
| ABRASIVE | |
| DENTAL TYPE SILICA (ZEODENT 165) | 3.00 |
| SYNTH. AMORPHOUS | |
| MICA | 0.70 |
| FILM COMPRISING PIGMENT BLUE 15 | 0.20 |
| TRICLOSAN | 0.30 |
| FLAVOR | 1.00 |
| Total | 100.00 |

This formulation is tested for whether the toothbrush bristles are stained following use. Whereas the original formulation showed substantial bristle staining (ΔE>20), the optimized formulation, while providing a good color change upon brushing, showed very little bristle staining (ΔE<5).

As those skilled in the art will appreciate, numerous changes and modifications may be made to the embodiments described herein without departing from the spirit of the invention. It is intended that all such variations fall within the scope of the appended claims.

1. A dentifrice having a pigment system which comprises one or more pigments, and titanium dioxide, wherein the level of titanium dioxide is 0.05-0.2% by weight of the dentifrice, wherein the dentifrice comprises Pigment Blue 15: 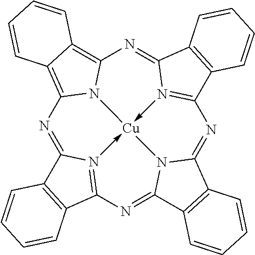
and wherein the dentifrice comprises 1.5-2.0% of sodium lauryl sulfate and 0.5 2.0% of a copolymer of methyl vinyl ether and maleic anhydride.

2. The dentifrice of claim 1, wherein the one or more pigments are releasable during brushing with an oral care implement.

3. The dentifrice of claim 1, wherein the releasable pigment provides a color signal of adequate brushing to the user and the titanium dioxide reduces or inhibits the staining of the oral care implement by the releasable pigment.

4. The dentifrice of claim 1, wherein a dissolvable or disintegratable film comprises the one or more releasable pigments which are released during brushing.

5. The dentifrice of claim 1 wherein the one or more releasable pigments comprises an insoluble pigment.

6. The dentifrice of claim 1 comprising
a) about 0.1% titanium dioxide
b) about 0.3% triclosan,
c) 1.5-2% sodium lauryl sulfate (SLS),
d) about 0.24% sodium fluoride,
e) 45-75% humectants comprising one or more of glycerin, sorbitol, and propylene glycol,
f) 1-10 thickeners comprising one or more of xanthan gum, carrageenan, and colloidal silica.
g) 0.5-3% of a copolymer of methyl vinyl ether and maleic anhydride, and
h) 15-25% water.

7. The dentifrice of claim 1, wherein the releasable pigment is substantially released after a period of greater than 30 seconds and less than 180 seconds of brushing in the presence of water, wherein the release of the releasable pigment provides a color signal to the user of adequate brushing.

8. A method of cleaning teeth with the dentifrice of claim 1.

9. The method of claim 8, wherein the pigment provides a color signal of adequate brushing with an oral care implement to the user and the titanium dioxide reduces or inhibits the staining of the oral care implement by the releasable pigment.

10. The method of claim 9, wherein the brushing time before the film dissolves or disintegrates is between 30 and 180 seconds.

11. The method of claim 9, wherein the oral care implement is as toothbrush and the titanium dioxide reduces or inhibits staining bristles of the toothbrush during brushing.
